(19) United States
(12) Patent Application Publication
(10) Pub. No.: US 2024/0102111 A1
(43) Pub. Date: Mar. 28, 2024

(54) MOLECULAR MARKER, SPECIFIC PRIMER PAIR AND IDENTIFICATION METHOD OF THE HIGH-QUALITY GANODERMA LUCIDUM STRAIN HMGIM-M624

(71) Applicants: INFINITUS (CHINA) COMPANY LTD., Guangdong (CN); INSTITUTE OF MICROBIOLOGY, GUANGDONG ACADEMY OF SCIENCES (GUANGDONG DETECTION CENTER OF MICROBIOLOGY, Guangdong (CN)

(72) Inventors: Xiuying KOU, Guangdong (CN); Xiaoxian WU, Guangdong (CN); Jian TANG, Guangdong (CN); Qingping WU, Guangdong (CN); Huiping HU, Guangdong (CN); Xiaowei LIANG, Guangdong (CN); Manjun CAI, Guangdong (CN); Yizhen XIE, Guangdong (CN); Yuanchao LIU, Guangdong (CN); Lijun ZHUO, Guangdong (CN); Ao WANG, Guangdong (CN); Na DU, Guangdong (CN)

(73) Assignees: INFINITUS (CHINA) COMPANY LTD., Guangdong (CN); INSTITUTE OF MICROBIOLOGY, GUANGDONG ACADEMY OF SCIENCES (GUANGDONG DETECTION CENTER OF MICROBIOLOGY, Guangdong (CN)

(21) Appl. No.: 18/066,282

(22) Filed: Dec. 15, 2022

(30) Foreign Application Priority Data
Sep. 23, 2022 (CN) 202211167492.4

(51) Int. Cl. C12Q 1/6895

(52) U.S. Cl. CPC C12Q 1/6895; C12Q 2600/16

(57) ABSTRACT

The present invention relates to a molecular marker, a specific primer pair, and an identification method of the high-quality Ganoderma lucidum strain HMGIM-M624. The high-quality Ganoderma lucidum strain HMGIM-M624 was preserved in Guangdong Microbial Culture Collection Center (address: 5th Floor, No. 59 Building of No. 100 Yard, Mid. Xianlie Road, Guangzhou City) with the preservation number of GDMCC No: 60889 on Nov. 7, 2019. The molecular marker is an InDel molecular marker. The high-quality Ganoderma lucidum strain HMGIM-M624 has a base deletion of CATGCTGTA at the 246451^{th}-246460^{th} site of the chromosome sca34. The present invention provides reagents for detecting the molecular marker of the high-quality Ganoderma lucidum strain HMGIM-M624. The reagents can distinguish the high-quality Ganoderma lucidum strain HMGIM-M624 from the other 11 G. lucidum strains for commercial cultivation, thus specifically identifying the high-quality Ganoderma lucidum strain HMGIM-M624.

### CROSS-REFERENCE TO RELATED APPLICATION

This application claims the priority benefit of China application serial no. 202211167492.4, filed on Sep. 23, 2022. The entirety of the above-mentioned patent application is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Dec. 5, 2022, is named Sequence Listing and is 4,531 bytes in size.

### BACKGROUND

### Technical Field

The present invention belongs to the technical field of molecular biology, and it particularly relates to a molecular marker, a specific primer pair, and an identification method of the high-quality Ganoderma lucidum strain HMGIM-M624.

### Description of Related Art

Ganoderma lucidum (Curtis) P. Karst. is a kind of fungus from Ganoderma P. Karst., Polyporaceae, Agaricomycetes. Since G. lucidum was recorded in Shen Nong's Herbal Classic, there are more and more folklores about G. lucidum and pharmaceutical values thereof have been continuously proven. G. lucidum, one of the most important medical fungi, has more than two thousand years of records and application history in China. In recent years, especially since G. lucidum has been recorded as a statutory traditional Chinese medicinal material in Pharmacopoeia of the People's Republic of China in 2000, researchers carry out extensive and in-depth studies to furtherly confirm the pharmacological basis and effects of G. lucidum. At present, it has been determined that G. lucidum fruiting body contains 9 categories of 300 kinds of compounds above, containing a wide variety of bioactive molecules, such as triterpenoids, proteins, polypeptides, nucleosides, furans, sterol, alkaloids, and amino acids. Among them, Polysaccharides and triterpenes are two major physiologically active constituents in G. lucidum. G. lucidum has many physiological and health-care functions, including modulation of the immune system, anti-tumor, hepatoprotection, cardiovascular protection, reducing hypertension, sedative-hypnotic, anti-aging, hypoglycemic, anti-oxidation, and more. G. lucidum has significant efficacy and thus has been commercially cultivated on a large scale. The artificial cultivation of G. lucidum has become an important industry in China. Currently, the annual yield of the cultivation and processing industries of G. lucidum has reached more than 100 billion Yuan. The development of G. lucidum's cultivation and production depends largely on the quality of strains. The main strains used in the commercially cultivating regions for G. lucidum in China, like Shandong, Zhejiang, Jilin, Anhui, Fujian, and Shaanxi, are ‘Hanzhi’ cultivars (introduced from South Korea) and ‘Hunong No. 1’ cultivar (native variety).

The high-quality Ganoderma lucidum strain HMGIM-M624 is screened and domesticated from the wild-type G. lucidum strains. The Ganoderma lucidum HMGIM-M624 strain showed excellent biological characteristics of mycelia and agronomic traits of cultivation in the planting test. In conventional substitute cultivation, more polysaccharide is synthesized and accumulated in the Ganoderma lucidum HMGIM-M624's fruiting body than in other commercial cultivars. In addition to that, the Ganoderma lucidum HMGIM-M624 strain has features of less spore, which thus avoids the influences of a large amount of spore powder on the environment and machine during the sporulation period of G. lucidum, and stable genetic character. Therefore, it is suitable for commercially cultivating on a large scale, and satisfying the demands for the industrialization development of G. lucidum.

The popularization of industrialized production comes with a higher demand for cultivating strains, so there is an urgent need for more convenient, efficient, and accurate identification of strains to provide a strong guarantee for G. lucidum's production. Currently, the main molecular markers of Ganoderma lucidum strains are Restriction Fragment Length Polymorphism (RFLP), Random Amplified Polymorphic DNA (RAPD), Sequence Characterized Amplified Region (SCAR), Sequence-Related Amplified Polymorphism (SRAP), Simple Sequence Repeats (SSR) and isoenzyme analysis. RFLP uses a DNA probe with a radioisotopes marker to detect a particular cut, or uncut, fragment, and thus the operation is more complicated. RAPD uses arbitrary primers which bind to the nonspecific sites on the DNA and amplify the DNA, but it is less reproducible than other techniques because of its low annealing temperatures. SSR is relatively convenient, cheap, and reliable, but some amplified bands are not clear and there exist technical defects, such as pseudoallele and amorph which cause errors in genotyping and data analysis.

Therefore, it is quite necessary to research a novel, convenient, accurate and efficient method suitable to specifically identify the high-quality Ganoderma lucidum strain HMGIM-M624.

### SUMMARY

The technical problem of the present invention is to overcome the shortcomings of the existing methods for specifically identifying the high-quality Ganoderma lucidum strain HMGIM-M624, and provide a molecular marker, a specific primer pair and an identification method for it.

The rapid development of molecular biotechnology, especially, the establishment and maturity of genomic sequencing technology provide effective means to develop a convenient, rapid, and accurate identification technology. As the third generation of a novel high-throughput and low-cost type of molecular marker, the InDel molecular marker can be obtained by bioinformatics methods and it is economical and applied, with the features of high specificity and good stability. The InDel molecular marker which is based on the insertion/deletion site on genomic sequence to design the specific primers, and then type the PCR product by DNA agarose gel electrophoresis, is quick, economical, and no need for complex experimental apparatus, and thus has strong operability. As a high-throughput molecular marker, the InDel marker's development process is based on sequence differences, and it has a lot of advantages such as high hereditary stability, extensive distribution, strong polymorphism, and strong universality, thus avoiding the problems of fuzzy subsequent analysis due to specificity and complexity, and the like. The present invention establishes an InDel molecular marker to identify the high-quality Ganoderma lucidum strain HMGIM-M624 rapidly and accurately and provides strain property rights protection for industrial applications.

The objective of the present invention is to provide a molecular marker of the high-quality Ganoderma lucidum strain HMGIM-M624.

The objective of the present invention is to further provide the use of the molecular marker in identifying the high-quality Ganoderma lucidum strain HMGIM-M624.

The objective of the present invention is to further provide reagents for detecting the high-quality Ganoderma lucidum strain HMGIM-M624.

The objective of the present invention is to further provide the use of the reagents in identifying the high-quality Ganoderma lucidum strain HMGIM-M624 or in preparing a detection kit of the high-quality Ganoderma lucidum strain HMGIM-M624.

The objective of the present invention is to further provide a kit for detecting the high-quality Ganoderma lucidum strain HMGIM-M624.

The objective of the present invention is to further provide the use of the kit in identifying the high-quality Ganoderma lucidum strain HMGIM-M624.

The objective of the present invention is to further provide a method for identifying the high-quality Ganoderma lucidum strain HMGIM-M624.

The above objectives of the present invention are achieved by the following technical means.

A molecular marker of the high-quality Ganoderma lucidum strain HMGIM-M624 is provided. The high-quality Ganoderma lucidum strain HMGIM-M624 described in the present invention was preserved in Guangdong Microbial Culture Collection Center (5th Floor, No. 59 Building of No. 100 Yard, Mid. Xianlie Road, Guangzhou City) with the preservation number of GDMCC No: 60889 on Nov. 7, 2019. The molecular marker described in the present invention is an InDel molecular marker with a base sequence (CATGCTGTA) deletion at the 24645^{th}-246460^{th} site of the chromosome sca34 of the high-quality Ganoderma lucidum strain HMGIM-M624. The accession number of the G. lucidum genome which contains the chromosome sca34 is PRJNA71455 in NCBI.

The use of a molecular marker in identifying the high-quality Ganoderma lucidum strain HMGIM-M624 is provided. The high-quality Ganoderma lucidum strain HMGIM-M624 described in the use of the molecular marker was preserved in Guangdong Microbial Culture Collection Center (address: 5^{th} Floor, No. 59 Building of No. 100 Yard, Mid. Xianlie Road, Guangzhou City) with the preservation number of GDMCC No: 60889 on Nov. 7, 2019.

Reagents for detecting the molecular marker of the high-quality Ganoderma lucidum strain HMGIM-M624 are provided. The high-quality Ganoderma lucidum strain HMGIM-M624 described in reagents for detecting the molecular marker was preserved in Guangdong Microbial Culture Collection Center (address: 5^{th} Floor, No. 59 Building of No. 100 Yard, Mid. Xianlie Road, Guangzhou City) with the preservation number of GDMCC No: 60889 on Nov. 7, 2019. The molecular marker described in the present invention is an InDel molecular marker with a base sequence (CATGCTGTA) deletion at the 246451^{th}-246460^{th} site of the chromosome sca34 of the high-quality Ganoderma lucidum strain HMGIM-M624. The accession number of the Ganoderma lucidum genome which contains the chromosome sca34 is PRJNA71455 in NCBI. The high-quality Ganoderma lucidum strain HMGIM-M624 contains the molecular marker.

Preferably, the reagents are a specific primer pair with the nucleotide sequences as shown in SEQ ID NO: 1 and SEQ ID NO: 2. The specific primer pair are used for amplifying DNA of the test samples, and only the sample of the high-quality Ganoderma lucidum strain HMGIM-M624 can obtain a 750 bp-900 bp band.

The specific primer pair with the nucleotide sequences as shown in SEQ ID NO: 1 and SEQ ID NO: 2 have an amplified product with the nucleotide sequence as shown in SEQ ID NO: 3.

The use of the reagents in identifying the high-quality Ganoderma lucidum strain HMGIM-M624 or in preparing a detection kit of the high-quality Ganoderma lucidum strain HMGIM-M624 is provided.

A kit for detecting the high-quality Ganoderma lucidum strain HMGIM-M624 is provided, and the kit contains the above reagents.

Preferably, the reagents contain the specific primer pair with the nucleotide sequences as shown in SEQ ID NO: 1 and SEQ ID NO: 2. The specific primer pair are used for amplifying DNA of the test samples, and only the sample of the high-quality Ganoderma lucidum strain HMGIM-M624 can obtain a 750 bp-900 bp band.

Preferably, the kit further contains PCR reaction reagents.

More preferably, the PCR reaction reagents are Taq PCR Master Mix buffer and double distilled water.

The use of the kit in identifying the high-quality Ganoderma lucidum strain HMGIM-M624 is provided.

An identifying method of the high-quality Ganoderma lucidum strain HMGIM-M624 is used to detect the above molecular marker. The high-quality Ganoderma lucidum strain HMGIM-M624 contains the above molecular marker. The high-quality Ganoderma lucidum strain HMGIM-M624 was preserved in Guangdong Microbial Culture Collection Center (address: 5^{th} Floor, No. 59 Building of No. 100 Yard, Mid. Xianlie Road, Guangzhou City) with the preservation number of GDMCC No: 60889 on Nov. 7, 2019.

Preferably, the identifying method includes steps of obtaining the genomic DNA of the G. lucidum test samples, serving the genomic DNA as a template, and performing PCR amplification by a specific primer pair with the nucleotide sequences as shown in SEQ ID NO: 1 and SEQ ID NO: 2. The sample with the 750 bp-900 bp amplified product is the high-quality Ganoderma lucidum strain HMGIM-M624.

Preferably, the reaction system of the PCR amplification is as follows: 25 μL of Taq PCR Master Mix buffer, 2 μL of the forward specific primer (10 μmol/L), 2 μL of the reverse specific primer (10 μmol/L), 1-2 μL of DNA template (250 mg/L), and 19-20 μL of double distilled water.

Further preferably, the reaction system of the PCR amplification is as follows: 25 μL of Taq PCR Master Mix buffer, 2 μL of the forward specific primer (10 μmol/L), 2 μL of the reverse specific primer (10 μmol/L), 2 μL of DNA template (250 mg/L), and 19 μL of double distilled water.

Preferably, the PCR amplification has the following procedures: step 1, amplification for 3-5 min at 90-95° C.; step 2, amplification for 30 s at 90-95° C.; step 3, amplification for 30 s at 58° C.; step 4, amplification for 20-55 s at 72° C.; step 5, amplification for 5-10 min at 72° C.; the steps 2-4 are cycled 30 times.

Further preferably, the PCR amplification has the following procedures: step 1, amplification for 3 min at 95° C.; step 2, amplification for 30 s at 95° C.; step 3, amplification for 30 s at 58° C.; step 4, amplification for 20 s at 72° C.; step 5, amplification for 10 min at 72° C.; the steps 2-4 are cycled 30 times.

Compared with the prior techniques, the present invention has the following beneficial effects.

The present invention provides a molecular marker of the high-quality Ganoderma lucidum strain HMGIM-M624. The molecular marker is an InDel molecular marker. The high-quality Ganoderma lucidum strain HMGIM-M624 has a base sequence (CATGCTGTA) deletion at the 246451^{th}-246460^{th} site of the chromosome sca34. The accession number of the Ganoderma lucidum genome which contains the chromosome sca34 is PRJNA71455 in NCBI. The present invention further provides reagents for detecting the high-quality Ganoderma lucidum strain HMGIM-M624. The reagents are a specific primer pair with the nucleotide sequences as shown in SEQ ID NO: 1 and SEQ ID NO: 2. The specific primer pair are used to amplify the DNA of test samples, and only the sample of the high-quality Ganoderma lucidum strain HMGIM-M624 can obtain a 750 bp-900 bp band. The reagents can be applied to distinguish the high-quality Ganoderma lucidum strain HMGIM-M624 from the other 11 G. lucidum strains for commercial cultivation, thus specifically identifying the high-quality Ganoderma lucidum strain HMGIM-M624. Therefore, the present invention can be used for the identification and protection of the high-quality Ganoderma lucidum strain HMGIM-M624. The identification method of the present invention is convenient, quick, and efficient, thus providing a new choice for the specificity identification of the high-quality Ganoderma lucidum strain HMGIM-M624.

### BRIEF DESCRIPTION OF THE DRAWINGS

FIG. 1 shows the agar gelatin electrophoresis results of ITS-PCR products of the high-quality Ganoderma lucidum strain HMGIM-M624 in Example 1 of the present invention and the other 11 G. lucidum strains for commercial cultivation, where M represents DL2000 DNA Marker, 1 represents the high-quality Ganoderma lucidum strain HMGIM-M624, and 2-12 represent the corresponding strains No. 2-12 in Table 1.

FIG. 2 shows the agar gelatin electrophoresis results of PCR products of the specific primer pair of the high-quality Ganoderma lucidum strain HMGIM-M624 in Example 2 of the present invention, where M represents DL2000 DNA Marker, 1 represents the high-quality Ganoderma lucidum strain HMGIM-M624, and 2-12 represent the corresponding strains No. 2-12 in Table 1.

FIG. 3 shows the agar gelatin electrophoresis results of PCR products of primer pairs W17-F/R, W18-F/R, W19-F/R, W21-F/R, W22-F/R and W23-F/R in Comparative Example of the present invention, where M represents DL2000 DNA Marker, 1 represents the high-quality Ganoderma lucidum strain HMGIM-M624, and 2-12 represent the corresponding strains No. 2-12 in Table 1.

### DESCRIPTION OF THE EMBODIMENTS

The present invention will be further described regarding the detailed examples, but the examples are not construed as limiting the present invention in any form. Reagents, methods and equipment used in the present invention are conventional reagents, methods and equipment in the technical field unless otherwise specified.

The reagents and materials used in the following examples are commercially available unless otherwise specified.

The high-quality Ganoderma lucidum strain HMGIM-M624 is screened and domesticated from the wild-type G. lucidum strains. The Ganoderma lucidum HMGIM-M624 strain showed excellent biological characteristics of mycelia and agronomic traits of cultivation in the planting test. In conventional substitute cultivation, more polysaccharide is synthesized and accumulated in the Ganoderma lucidum HMGIM-M624's fruiting body than in other commercial cultivars. In addition to that, the Ganoderma lucidum HMGIM-M624 strain has features of less spore, which thus avoids the influences of a large amount of spore powder on the environment and machine during the sporulation period of G. lucidum, and stable genetic character. Therefore, it is suitable for commercially cultivating on a large scale, and satisfying the demands for the industrialization development of G. lucidum.

The high-quality Ganoderma lucidum strain HMGIM-M624 was preserved in Guangdong Microbial Culture Collection Center (address: 5^{th} Floor, No. 59 Building of No. 100 Yard, Mid. Xianlie Road, Guangzhou City) with the preservation number of GDMCC No: 60889 on Nov. 7, 2019. The strain has been published in a Chinese patent for invention

The ITS identification result of the high-quality Ganoderma lucidum strain HMGIM-M624 is a Ganoderma lingzhi strain. G. lucidum recorded in Pharmacopoeia of the People's Republic of China is a dried fruiting body of the fungus G. lucidum or G. sinense of Polyporaceae. However, with the progress of phylogeny research, professor Dai Yucheng, a taxonomy specialist in China, has shown that the G. lucidum widely distributed and cultivated in China is different from the G. lucidum originated in Europe in 2013 research. Wasser et al. (2006) and Wasser (2011) also have indicated that G. lucidum is mistakenly applied in the medicinal fungus G. lingzhi. In fact, Chinese G. lucidum (commonly known as Chizhi) is a new species and named G. lingzhi S. H. Wu, Y. Cao & Y. C. Dai (Cao et al. 2012). Therefore, the Latin name of G. lucidum recorded in Pharmacopoeia of the People's Republic of China should be G. lingzhi instead of G. lucidum. The G. lucidum strain HMGIM-M624 marked in this patent is a wild strain of local Chinese G. lingzhi. However, G. lucidum is still used as the Latin name of G. lingzhi in Pharmacopoeia of the People's Republic of China. Therefore, the species name of the G. lucidum strain HMGIM-M624 marked in the present application is tentatively determined as G. lucidum M624 and would be corrected after the Pharmacopoeia of the People's Republic of China is revised.

### Example 1 Obtaining of Strain InDel Sites

1. Extraction of the Genomic DNA of G. lucidum Strains

The high-quality Ganoderma lucidum strain HMGIM-M624 was chosen from the germplasm bank of G. lucidum established by the inventor's team as an experimental strain, and the other 11 G. lucidum strains for commercial cultivation were used as control strains. The strain information is shown in Table 1.

TABLE 1

| Strain | Strain serial | | |
|---|---|---|---|
| number | number | Strain name | Strain source |
| 1 | HMGIM-M624 | HMGIM-M624 | The team obtained G. lucidum |
| | | (Ganoderma lucidum) | strain HMGIM-M624 by the |
| | | | artificial domestication of the |
| | | | wild high-quality G. lucidum |
| | | | strain which was collected on the |
| | | | field in Lu'an City of Anhui Province. |
| 2 | GL-AH | Anhui | Anhui G. lingzhi is a fine species |
| | | Ganoderma lingzhi | cultivated locally, small-scale |
| | | (Ganoderma lingzhi) | cultivated in Anhui areas and has |
| | | | high and stable yield. |
| 3 | MC-GL-0054 | Hunong No. 1 | Hunong No. 1 G. lingzhi is a |
| 4 | MC-GL-0056 | Ganoderma lingzhi | species cultivated locally and |
| 5 | MC-GL-0057 | (Ganoderma lingzhi) | bred by a postgraduate of |
| | | | Institute of Edible Fungi, |
| | | | Shanghai Academy of |
| | | | Agricultural Sciences, and the |
| | | | species has been promoted and |
| | | | cultivated for many years in |
| | | | Anhui, Zhejiang, Fujian, |
| | | | Shanghai and many other places, |
| | | | and is a Ganoderma lucidum |
| | | | strain with more stable |
| | | | cultivation and mature technology. |
| 6 | MC-GL-0052 | Longzhi No. 1 | Longzhi No. 1 G. lingzhi is a |
| | | Ganoderma lingzhi | species cultivated locally, studied |
| | | (Ganoderma lingzhi) | and bred jointly by Longquan |
| | | | Xinglong Biotechnology Co., |
| | | | Ltd., Zhang Liangming Strains |
| | | | Field and Zhejiang Academy of |
| | | | Agricultural Sciences, and has |
| | | | been promoted and cultivated in |
| | | | many places of Longquan City, |
| | | | Zhejiang Province. |
| 7 | MC-GL-0072 | Quanzhi No. 9 | Quanzhi No. 9 G. lingzhi is a |
| | | Ganoderma lingzhi | species cultivated locally, and |
| | | (Ganoderma lingzhi) | has been promoted and cultivated |
| | | | in many places of Quanzhou |
| | | | City, Fujian Province. |
| 8 | MC-GL-0058 | Chizhi No. 9 | Chizhi No. 9 Ganoderma lingzhi |
| | | Ganoderma lingzhi | is separated from fruiting body |
| | | (Ganoderma lingzhi) | tissues of G. lingzhi |
| | | | commercially available. |
| 9 | MC-GL-0047 | Hanzhi | It is a species introduced from |
| 10 | MC-GL-0053 | Ganoderma lingzhi | South Korea, and has been |
| 11 | MC-GL-0060 | (Ganoderma lingzhi) | promoted and cultivated in |
| 12 | MC-GL-0063 | | Anhui, Zhejiang, Fujian, |
| | | | Shanghai and many other places |
| | | | for many years, and is a |
| | | | Ganoderma lucidum strain with |
| | | | more stable cultivation and |
| | | | mature technology. |

The high-quality Ganoderma lucidum strain HMGIM-M624 and the other 11 Ganoderma lucidum strains for commercial cultivation, which were preserved at low temperature, were activated at 25° C. and then transferred onto the PDA plates covered with cellophane film for culture 5-7 days at 25° C. in the dark, thus obtaining fresh mycelia. A proper amount of mycelia were collected into a grinding tube with sterile forceps to extract the genomic DNA of the mycelia using a full-automatic nucleic acid isolation machine matched with a kit for extracting fungal genomic DNA with immunomagnetic beads (Guangzhou Mabio Biotechnology Co., Ltd., Art. No.: DNF628-05B [the customized version]). The obtained DNA solution (DNA template) was refrigerated at −20° C. for further use.

Genomic DNA of the extracted mycelia served as a template and a universal primer pair of the internal transcribed spacer (ITS) of fungal ribosome ITS1(TCCGTAGGTGAACCTGCGG)/ITS4(TCCTCCGCTTATTGATATGC) was used for ITS-PCR experiment. The amplification was performed on a Biometra PCR instrument. ITS-PCR reaction system (50 μl in total) is shown in Table 2 and the ITS-PCR amplification procedures are shown in Table 3 below.

TABLE 2
ITS-PCR reaction system

| | Amount |
|---|---|
| Composition | (μL) |
| Taq PCR Master Mix buffer (Sangon B110006) | 25 |
| ITS1 (10 μmol/L) | 2 |
| ITS4 (10 μmol/L) | 2 |
| Template DNA | 2 |
| Double distilled water | 19 |

TABLE 3
ITS-PCR amplification procedure

| Step | Temperature | Time | |
|---|---|---|---|
| 1 | 95° C. | 3 | min |
| 2 | 95° C. | 30 | s |
| 3 | 56° C. | 30 | s |
| 4 | 72° C. | 1 | min |

Steps 2-4 were cycled 30 times

| | | | |
|---|---|---|---|
| 5 | 72° C. | 10 | min |

10 μL of the PCR products and DNA Marker were applied on a 10% (mass-volume ratio) agarose gel for electrophoresis for 30 min at 80 V, and then the agarose gel was placed onto a gel imager for observation.

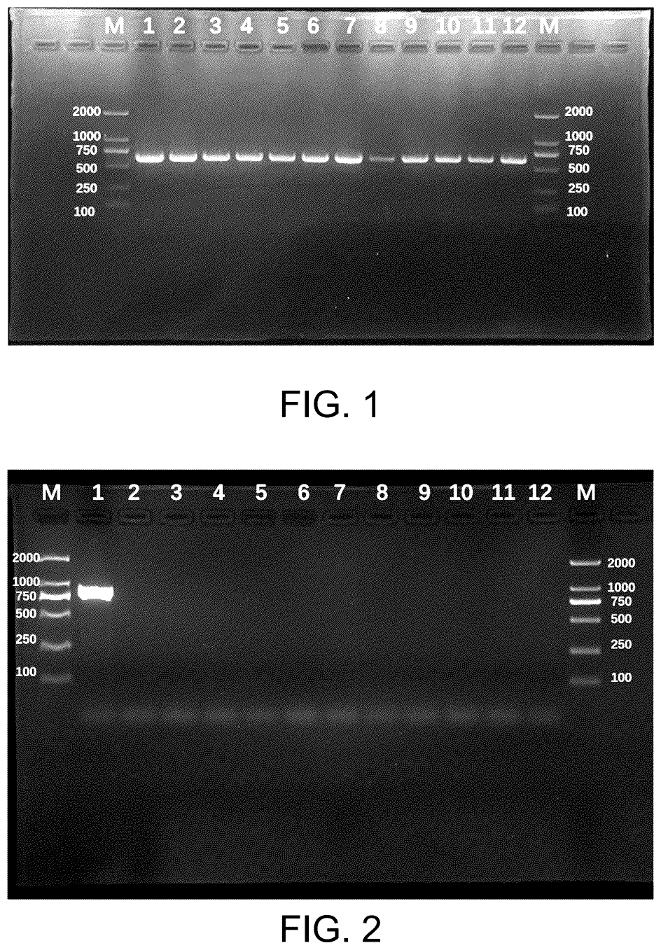

The agar gelatin electrophoresis results of ITS-PCR products of the high-quality Ganoderma lucidum strain HMGIM-M624 and the other 11 G. lucidum strains for commercial cultivation are shown in FIG. 1, where M represents DL2000 DNA Marker, 1 represents the high-quality Ganoderma lucidum strain HMGIM-M624, and 2-12 represent the corresponding strains No. 2-12 in Table 1.

FIG. 1 shows that both the ITS-PCR products of the high-quality Ganoderma lucidum strain HMGIM-M624 and the other 11 G. lucidum strains for commercial cultivation have bands, indicating free of quality problem in the extracted DNA.

2. Whole Genome Resequencing

After the sample genomic DNA was detected and determined as G. lucidum, the DNA sequence was fragmentated by ultrasonic wave to form random fragments, and then repaired the end, added “A” bases to the 3′-terminal ligated the sequencing adapters, enriched the random fragments about 400 bp by magnetic beads adsorption, and formed the sequencing library by PCR amplification. After library quality control, the qualified library was sequenced by using Illumina HiSeq™ platform. The sequencing strategy was Illumina PE150 and the total sequencing reads were 300 bp. Made quality evaluation and filtering for the raw reads (Paired ends) obtained from sequencing to get Clean Reads, which could be used for subsequent bioinformatics. Aligned Clean Reads with the reference genome of G. lucidum (Accession: PRJNA71455) by BWA software, thus obtaining the home location of the sequence (namely, BAM file). The BAM file was corrected by the Best Practices process of GATK, and the detection of Small InDel markers was carried out. SNPEff software and gene prediction information of the reference genome were applied for variation function annotation to obtain the functional annotation information of InDel. The Best Practices process of GATK was applied to process the alignment result (BAM file), and Haplotyper method of GATK was applied for InDel detection and filtering. The filtering conditions were subjected to the parameters recommended by GATK, and the project sample was subjected to develop the InDel marker.

The whole genome was resequenced to obtain 183G Clean Data in total, and the sequencing Q30 was 93.84% and GC content was 52.81%. 118648 InDel sites were obtained by bioinformatics analysis. The length of the insertion/deletion fragments was mainly focused on within 10 bp.

### Example 2 Obtaining and PCR Identification of the Specific Primer Pair

1. Method

(1) Specific Primer Pair

Based on the InDel detection results of the whole genome resequencing in Example 1, the sequencing depth and the base number of the insertion/deletion fragments, the specific InDel site of the high-quality Ganoderma lucidum strain HMGIM-M624 was screened and the corresponding specific primer pair was designed; the genomic DNA of the high-quality Ganoderma lucidum strain HMGIM-M624 and the other 11 G. lucidum strains for commercial cultivation served as templates for PCR screening and verification.

By alignment to the reference genome of G. lucidum, it is found that the high-quality Ganoderma lucidum strain HMGIM-M624 has a base sequence (CATGCTGTA) deletion at the 24645^{th}-246460^{th} site of the chromosome sca34. The accession number of the G. lucidum genome which contains the chromosome sca34 is PRJNA71455 in NCBI. The specific primer pair corresponding to the site InDel is as follows.

W20-F:
(SEQ ID NO: 1)
GCCAATCGTCAACATCGCTC,
W20-R:
(SEQ ID NO: 2)
TCGACAACCCTTGGTCTTCG.

(2) PCR Identification

Genomic DNA extracted from the high-quality Ganoderma lucidum strain HMGIM-M624 in Table 1 of Example 1 and the other 11 G. lucidum strains for commercial cultivation served as templates, and the specific primer pair W20-F (SEQ ID NO: 1)/W20-R (SEQ ID NO: 2) was subjected to PCR amplification. The PCR reaction system is shown in Table 4 below, and the PCR amplification procedures are shown in Table 5 below.

TABLE 4
PCR reaction system

| | Amount |
|---|---|
| Composition | (μL) |
| Taq PCR Master Mix buffer (Sangon B110006) | 25 |
| W20-F (10 μmol/L) (SEQ ID NO: 1) | 2 |
| W20-R (10 μmol/L) (SEQ ID NO: 2) | 2 |
| Template DNA | 2 |
| Double distilled water | 19 |

TABLE 5
PCR amplification procedure

| Step | Temperature | Time | |
|---|---|---|---|
| 1 | 95° C. | 3 | min |
| 2 | 95° C. | 30 | s |
| 3 | 58° C. | 30 | s |
| 4 | 72° C. | 20 | s |

Steps 2-4 were cycled 30 times

| | | | |
|---|---|---|---|
| 5 | 72° C. | 10 | min |

10 μL of the PCR products and DNA Marker were applied on a 2% (mass-volume ratio) agarose gel for electrophoresis for 30 min at 80 V, and then the agarose gel was placed onto a gel imager for observation.

2. Result

The agar gelatin electrophoresis result of the PCR products of the specific primer pair of the high-quality Ganoderma lucidum strain HMGIM-M624 are shown in FIG. 2, where M represents DL2000 DNA Marker, 1 represents the high-quality Ganoderma lucidum strain HMGIM-M624, and 2-12 represent the corresponding strains No. 2-12 in Table 1.

FIG. 2 shows that only the high-quality Ganoderma lucidum strain HMGIM-M624 has a specific band at 750 bp-900 bp, and the other 11 G. lucidum strains for commercial cultivation have no band. The specific primer pair W20-F/W20-R may be used to distinguish the high-quality Ganoderma lucidum strain HMGIM-M624 from the other 11 G. lucidum strains for commercial cultivation.

The remaining PCR products were sent for detection for bi-directional sequencing which was completed by Beijing Genomics Institute (BGI). Amplified sequences of the specific primer pair W20-F/W20-R of the high-quality Ganoderma lucidum strain HMGIM-M624 were obtained by sequencing, and the amplified specific nucleotide sequence was 864 bp in total. The sequence is as follows.

(SEQ ID NO: 3)
GGGCAGTTGCGATCCATGAGGCGGAACCTTGCCCCAGAGCTCCGAG
TCCACCTGCGTCTCCTCGTCCCACGTCGCCCTGAAGCTCGGTCGCG
TGTGGACGTCCTCCGTGTACCGCCGCAGGCGCGCGAAGCGCGGCCC
ACCCTGCTCGAGCGGGTTCTCGCCCATGTCGTGCGTGAGGAACATG
AACGTGCGCGCGAGGAATGGCGCGATGGTCAATGTCCGCGATCGAC
CAGCGGCCAGAGAGGAGCGTCTGGCGCGCCTCGAGGGCATCAAGGA
ACTTGGCGCGCGGGGCGCCGCGGAAGAAGAAATCGAGGAAGGCGTC
GGGGAGGACGCGCGTCCGAGTCTCGAACGTGTGGATGAACAACCGC
GCGTGGGCGCACAGGACGGAACTGGCTGGGAGGAGGGAAGCGGAGA
GGTCATGATAGAGGTCAGCAAGGACGAGGACGAGGGTGAGGAACTC
GAGGAGCACGAGCGACTCGGCGAGCTTCACGGAGGAGGGCCAAGGC
TGGTGTGCGGCGGGAACGGCGGGTCGTCCGTAGGTGATTGCGGGGG
TCATCTGGGTCTGCGGGTTGCGTCAGCGCACGAGGGGGAGCGAGTG
GGATGTATGCGTGGTGGGGAGCGGGAAAGCTGACCATATCATGTCG
ACGGGGCTGGCGTCGGAGACGAACCAGGAGGGTTTGTTTTGAGGGT
CGATGGTGTGCGGGATGTATTCTGCGTTCGCTTCTTCGAGGGCGAT
GTGCACTCGGCGTGCGTAGGGTGAGTACTGCACGGGCGTGAGTCAC
TGCGGAGAGACTAGAGCACGTTCCCAGAAAGGCGAACAGAGAACTC
ACTACACGGGTATAGAAGCTGATGCGGAGGCATTGT.

The size of the amplified specific nucleotide sequence was the same as that of the agar gelatin electrophoretic band.

The length of the specific band may vary from different sequencers, but be within 750 bp to 900 bp.

To sum up, the specific primer pair W20-F (SEQ ID NO: 1)/W20-R (SEQ ID NO: 2) in the present invention may be used to distinguish the high-quality Ganoderma lucidum strain HMGIM-M624 from the other 11 Ganoderma lucidum strains for commercial cultivation. The above result indicates that the primer pair have strong specificity and selectivity to the high-quality Ganoderma lucidum strain HMGIM-M624.

### Comparative Example

1. Method

(1) Sites and Primer Pairs

Based on the InDel detection results of the whole genome resequencing, by the alignment to the reference genome of G. lucidum (Accession: PRJNA71455) and according to the sequencing depth and the base number of insertion/deletion fragments, 6 specific InDel sites W17, W18, W19, W21, W22 and W23 of the high-quality Ganoderma lucidum strain HMGIM-M624 were further screened. The site information is shown in Table 6. The corresponding specific primer pairs were designed according to the 6 InDel sites screened. The primer information is shown in Table 7.

TABLE 6
Site information

| | Variation | |
|---|---|---|
| Site | type | Details of the variation site |
| W17 | Base | Insertion of the fragment |
| | fragment | “CGCGGCGGCA” between the |
| | insertion | 1811834th site and the |
| | | 1811835th site of the |
| | | chromosome sca4 |
| W18 | Base | Deletion of the fragment |
| | fragment | “GCGGCGGCA” from the base |
| | deletion | “CTGTCTCAGGT” at the 1082129th |
| | | site to the 1082139th site |
| | | of the chromosome sca6 |
| W19 | Base | Insertion of the fragment |
| | fragment | “TCTCCGAGGG” between the |
| | insertion | 329174th site and the |
| | | 329175th site of the |
| | | chromosome sca12 |
| W21 | Base | Insertion of the fragment |
| | fragment | “GTCCTCCTCCT” between the |
| | insertion | 414158th site and the 414159th |
| | | site of the chromosome sca25 |
| W22 | Base | Insertion of the fragment |
| | fragment | “TCATCCACCA” between the |
| | insertion | 191185th site and the 191186th |
| | | site of the chromosome sca35 |
| W23 | Base | Insertion of the fragment |
| | fragment | “TCCCTCCACCG” between the |
| | insertion | 89033rd site and the 89034th |
| | | site of the chromosome sca36 |

TABLE 7
Primer information

| Site | Primer sequence |
|---|---|
| W17 | F: GCTTGTCACTGTGCAGGTTG |
| | R: GAGAGGCCGATGATCTGGTG |
| W18 | F: CCTGTTTGATTCGGGACCCA |
| | R: GACTCGGACAGCGTGGATAG |
| W19 | F: GAGTATGTACTCGACGCCCG |
| | R: GTTGCACGTCGTAATGTCCG |
| W21 | F: GAAGGACTGGCTCTGGTGAC |
| | R: TGGCGATTTGAGGCTGCTAA |
| W22 | F: ACGGGCATACAACGCAACTA |
| | R: AGCGTTGAAGTCAAGAGCGA |
| W23 | F: AGAGAGAGCACGGACCTGAT |
| | R: AACGTGTACCACATCCCGAC |

(2) PCR Identification

Genomic DNA extracted from the high-quality Ganoderma lucidum strain HMGIM-M624 in Table 1 of Example 1 and the other 11 Ganoderma lucidum strains for commercial cultivation served as templates. Primer pairs W17-F/R, W18-F/R, W19-F/R, W21-F/R, W22-F/R and W23-F/R were respectively subjected to PCR amplification. The PCR reaction system is the same as that in Table 4 of Example 2, and the difference lies in that the primer pair W20-F/R is respectively replaced by W17-F/R, W18-F/R, W19-F/R, W21-F/R, W22-F/R or W23-F/R. PCR amplification procedure is referred to Table 5 of Example 2, wherein in step 3, the corresponding temperature of the primer pairs W17-F/R, W18-F/R, W19-F/R, and W22-F/R is 58° C. The corresponding temperature of the primer pairs W21-F/R and W23-F/R is 59° C. The rest steps are the same as those in Table 5.

10 μL of the PCR products and DNA Marker were applied on a 2% (mass-volume ratio) agarose gel for electrophoresis for 30 min at 80 V, and then the agarose gel was placed onto a gel imager for observation.

2. Result

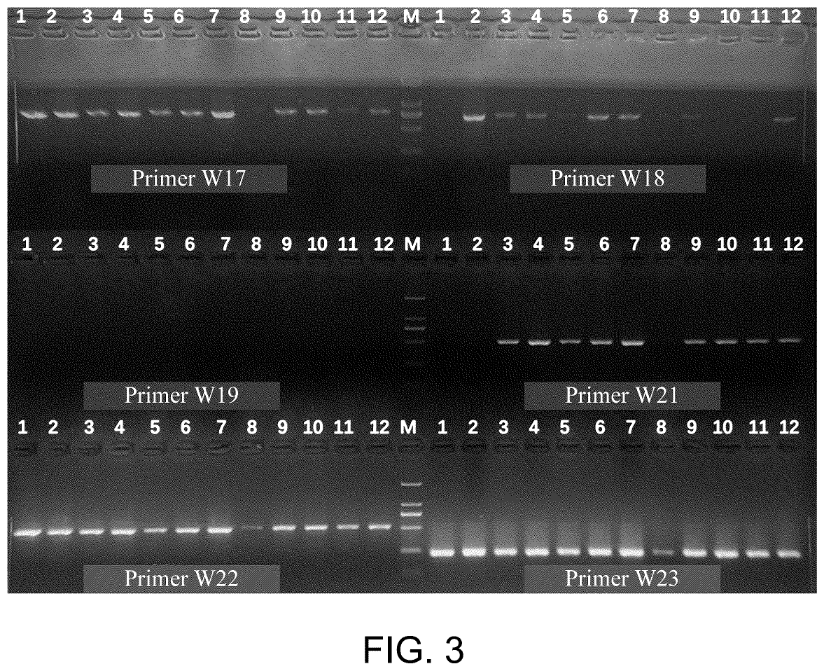

The agar gelatin electrophoresis results of PCR products of the primer pairs W17-F/R, W18-F/R, W19-F/R, W21-F/R, W22-F/R and W23-F/R are shown in FIG. 3, where M represents DL2000 DNA Marker, 1 represents the high-quality Ganoderma lucidum strain HMGIM-M624, and 2-12 represent the corresponding strains No. 2-12 in Table 1.

FIG. 3 shows that 6 pairs of primer W17-F/R, W18-F/R, W19-F/R, W21-F/R, W22-F/R and W23-F/R may not specifically distinguish the high-quality Ganoderma lucidum strain HMGIM-M624 from the other 11 Ganoderma lucidum strains for commercial cultivation, indicating that the InDel sites W17, W18, W19, W21, W22 and W23 as well as the primer pairs W17-F/R, W18-F/R, W19-F/R, W21-F/R, W22-F/R and W23-F/R thereof are not suitable for the specificity identification of the high-quality Ganoderma lucidum strain HMGIM-M624.

### Example 3 Kit for Detecting a Molecular Marker of the High-Quality Ganoderma lucidum Strain HMGIM-M624

A kit for detecting the molecular marker of the high-quality Ganoderma lucidum strain HMGIM-M624 contains Taq PCR Master Mix buffer, the specific primer pair with the nucleotide sequences as shown in SEQ ID NO: 1 and SEQ ID NO: 2, template DNA and double distilled water.

The genomic DNA of the Ganoderma lucidum sample to be detected served as template DNA. The Taq PCR Master Mix buffer, the specific primer pair with the nucleotide sequences as shown in SEQ ID NO: 1 and SEQ ID NO: 2, and double distilled water in the kit were subjected to PCR amplification according to the reaction system as shown in Table 4 of Example 2 and the amplification procedure as shown in Table 5. If the amplified product has a specific band at 750 bp-900 bp, the strain is the high-quality Ganoderma lucidum strain HMGIM-M624.

### Example 4 Method for Identifying the High-Quality Ganoderma lucidum Strain HMGIM-M624

In the method for identifying the high-quality Ganoderma lucidum strain HMGIM-M624, genomic DNA of a G. lucidum samples were extracted as templates, and the specific primer pair with the nucleotide sequences as shown in SEQ ID NO: 1 and SEQ ID NO: 2 were subjected to PCR amplification. The PCR reaction system is the same as that in Table 4 of Example 2; and the amplification procedure is the same as that in Table 5 of Example 2.

10 μL of the PCR products and DNA Marker were applied on a 2% (mass-volume ratio) agarose gel for electrophoresis for 30 min at 80 V, and then the agarose gel was placed onto a gel imager for observation. If the PCR product has a specific band at 750 bp-900 bp by agar gelatin electrophoresis, the strain is the high-quality Ganoderma lucidum strain HMGIM-M624.

The above examples are preferred embodiments of the present invention but are not construed as limiting the embodiments of the present invention. Any other changes, modifications, replacements, combinations and simplifications made within the spirit and principle of the present invention shall be equivalent substitution modes and shall fall within the protection scope of the present invention.

What is claimed is:

1. A molecular marker of a high-quality Ganoderma lucidum strain HMGIM-M624, wherein the high-quality Ganoderma lucidum strain HMGIM-M624 was preserved in Guangdong Microbial Culture Collection Center (address: 5^{th} Floor, No. 59 Building of No. 100 Yard, Mid. Xianlie Road, Guangzhou City) with a preservation number of GDMCC No: 60889 on Nov. 7, 2019;
wherein the molecular marker is an InDel molecular marker, and the high-quality Ganoderma lucidum strain HMGIM-M624 has a base deletion of CATGCTGTA at the 246451^{th}-246460^{th} site of the chromosome sca34,
wherein an accession number of the Ganoderma lucidum genome which contains the chromosome sca34 is PRJNA71455 in NCBI.

2. A use of the molecular marker of claim 1 in identifying the high-quality Ganoderma lucidum strain HMGIM-M624, wherein the high-quality Ganoderma lucidum strain HMGIM-M624 is stated in claim 1.

3. A reagent for detecting a molecular marker of a high-quality Ganoderma lucidum strain HMGIM-M624, wherein the high-quality Ganoderma lucidum strain HMGIM-M624 is stated in claim 1, and the high-quality Ganoderma lucidum strain HMGIM-M624 contains the molecular marker of claim 1.

4. The reagent according to claim 3, wherein the reagent is a specific primer pair with the nucleotide sequences as shown in SEQ ID NO: 1 and SEQ ID NO: 2.

5. A use of the reagent of claim 3 in identifying the high-quality Ganoderma lucidum strain HMGIM-M624 of claim 3 or in preparing a detection kit of the high-quality Ganoderma lucidum strain HMGIM-M624 of claim 3.

6. A use of the reagent of claim 4 in identifying the high-quality Ganoderma lucidum strain HMGIM-M624 of claim 4 or in preparing a detection kit of the high-quality Ganoderma lucidum strain HMGIM-M624 of claim 4.

7. A kit for detecting a high-quality Ganoderma lucidum strain HMGIM-M624, wherein the kit contains the reagent of claim 3.

8. A kit for detecting a high-quality Ganoderma lucidum strain HMGIM-M624, wherein the kit contains the reagent of claim 4.

9. A use of the kit of claim 7 in identifying the high-quality Ganoderma lucidum strain HMGIM-M624.

10. A use of the kit of claim 8 in identifying the high-quality Ganoderma lucidum strain HMGIM-M624.

11. A method for identifying a high-quality Ganoderma lucidum strain HMGIM-M624, comprising:
detecting the molecular marker of claim 1, wherein the high-quality Ganoderma lucidum strain HMGIM-M624 contains the molecular marker of claim 1, and the high-quality Ganoderma lucidum strain HMGIM-M624 was preserved in Guangdong Microbial Culture Collection Center (address: 5^{th} Floor, No. 59 Building of No. 100 Yard, Mid. Xianlie Road, Guangzhou City) with the preservation number of GDMCC No: 60889 on Nov. 7, 2019.

12. The method according to claim 11, further comprising:
obtaining genomic DNA of G. lucidum samples to be identified; and
serving the genomic DNA as templates, and performing PCR amplification by a specific primer pair with the nucleotide sequences as shown in SEQ ID NO: 1 and SEQ ID NO: 2.


SEQUENCE LISTING (3 sequences)

SEQ ID NO: 1 - DNA, 20 bp, synthetic construct
  source 1..20: mol_type = other DNA
gccaatcgtcaacatcgctc

SEQ ID NO: 2 - DNA, 20 bp, synthetic construct
  source 1..20: mol_type = other DNA
tcgacaacccttggtcttcg

SEQ ID NO: 3 - DNA, 864 bp, Ganoderma lucidum
  source 1..864: mol_type = genomic DNA
gggcagttgcgatccatgaggcggaaccttgccccagagctccgagtccacctgcgtctcctcgtcccacgtcgccctgaagctcggtcgcgtgtggacgtcctccgtgtaccgccgcaggcgcgcgaagcgcggcccaccctgctcgagcgggttctcgcccatgtcgtgcgtgaggaacatgaacgtgcgcgcgaggaatggcgcgatggtcaatgtccgcgatcgaccagcggccagagaggagcgtctggcgcgcctcgagggcatcaaggaacttggcgcgcggggcgccgcggaagaagaaatcgaggaaggcgtcggggaggacgcgcgtccgagtctcgaacgtgtggatgaacaaccgcgcgtgggcgcacaggacggaactggctgggaggagggaagcggagaggtcatgatagaggtcagcaaggacgaggacgagggtgaggaactcgaggagcacgagcgactcggcgagcttcacggaggagggccaaggctggtgtgcggcgggaacggcgggtcgtccgtaggtgattgcgggggtcatctgggtctgcgggttgcgtcagcgcacgagggggagcgagtgggatgtatgcgtggtggggagcgggaaagctgaccatatcatgtcgacggggctggcgtcggagacgaaccaggagggtttgttttgagggtcgatggtgtgcgggatgtattctgcgttcgcttcttcgagggcgatgtgcactcggcgtgcgtagggtgagtactgcacgggcgtgagtcactgcggagagactagagcacgttcccagaaaggcgaacagagaactcactacacgggtatagaagctgatgcggaggcattgt